(19) United States
(12) Patent Application Publication
(10) Pub. No.: US 2004/0126360 A1
(43) Pub. Date: Jul. 1, 2004

(54) Oral formulations for proteins and polypeptides

(76) Inventors: Mark C. Manning, Loveland, CO (US); Rajiv Nayar, Richmond, CA (US)

Correspondence Address: PERKINS COIE LLP, P.O. BOX 2168, MENLO PARK, CA, 94026, US

(21) Appl. No.: 10/683,214

(22) Filed: Oct. 7, 2003

Related U.S. Application Data
(60) Provisional application No. 60/417,292, filed on Oct. 9, 2002.

(51) Int. Cl. A61K 38/21

(52) U.S. Cl. 424/85.5; 424/85.6; 424/85.7

(57) ABSTRACT

Compositions for preparation of oral dosage forms for administration of proteins and polypeptides are described. The compositions include a species capable of stabilizing the protein or polypeptide such that it retains biological activity during storage, for activity upon administration in vivo.

This application claims priority to U.S. provisional patent application No. 60/417,292 filed on Oct. 9, 2002, which is incorporated herein in its entirety by reference.

### FIELD OF THE INVENTION

The present invention relates to a liquid formulation suitable for oral administration of proteins and polypeptides. More specifically, the invention provides a composition capable of stabilizing a protein or polypeptide, and in particular a cytokine, and more particularly, an interferon, for formulation into and storage of an oral dosage form.

### BACKGROUND OF THE INVENTION

Oral delivery of therapeutic agents is a preferred mode of administration due to its convenience and simplicity, both contributing to better patient compliance. Recombinant technology has made available a wider selection of proteins and polypeptides for use as therapeutic agents, and oral delivery of proteins and polypeptides is of increasing interest and value. However, because proteins and polypeptides can be unstable during storage, leading to loss of biological activity, an oral formulation is preferably designed to optimize stability for retention of activity during storage and upon administration.

Formulation factors that require consideration of design of an oral formulation of a protein or polypeptide include the solution behavior of the protein or polypeptide in aqueous and nonaqueous solvents and the effect of ionic strength, solution pH, and solvent type on the stability and structure of the protein or polypeptide. The effect of temperature during formulation on the stability and structure of the protein or polypeptide must also be considered, as should the overall suitability of the formulation for incorporation into an oral dosage form, and particularly into an oral liquid dosage form, such as a gelatin capsule or syrup.

### SUMMARY OF THE INVENTION

Accordingly, it is an object of the invention to provide a composition comprising a protein or a polypeptide formulated in an oral dosage form, where the protein or polypeptide is stabilized by a species present in the formulation for retention of biological activity.

It is another object of the invention to provide a method of preparing an oral dosage form for a protein or a polypeptide that stabilizes the protein or polypeptide so as to retain its biological activity.

It is a further object of the invention to provide a method for selecting a formulation capable of incorporation into an oral dosage form and capable of stabilizing a protein or a polypeptide for activity upon in vivo administration.

In one aspect, the invention relates to a composition comprising a protein or a polypeptide and a selected species effective to stabilize the polypeptide or protein at a selected pH. In one embodiment, the species is capable of stabilizing the polypeptide or protein by binding to the protein or polypeptide in order to preserve the protein's tertiary structure. Such binding can be virtually any type of binding, including but not limited to electrostatic, ionic, hydrogen, and covalent binding interactions.

In one embodiment, the protein or polypeptide is a cytokine. In a more preferred embodiment, the cytokine is an interferon. Preferred interferons include both type I and type II interferons, including but not limited to interferon-α, interferon-β, interferon-γ, interferon-ω, and interferon-τ.

The species in the composition effective to stabilize the protein or polypeptide, in one embodiment, is a buffer. In a preferred embodiment, the buffer is an amino acid, and a preferred amino acid is L-histidine.

The pH of the composition can range from between about 5-8, more preferably from 6-8, and most preferably from 6.9-7.3.

In another aspect, the invention includes a method of preparing an oral dosage form for a protein or polypeptide. The method includes mixing the protein or polypeptide with a species effective to stabilize the polypeptide or protein at a selected pH.

In still another aspect, the invention provides a method for selecting a dosage form composition for a protein or polypeptide that includes a selected species effective to stabilize the protein or polypeptide for biological activity upon in vivo administration. The selection method includes (1) selecting a protein or polypeptide for formulation; (2) preparing solutions of the selected protein or polypeptide in different buffers at different pH values and (3) measuring the effect of the buffer on the structure and stability of the protein. Buffers that result in a relatively small change in protein tertiary structure of alpha-helical proteins are selected as the species effective to stabilize the protein or polypeptide. The effect of the buffer on the protein's structure can be measured using various analytical techniques, such as spectroscopic techniques like UV circular dichroism.

These and other objects and features of the invention will be more fully appreciated when the following detailed description of the invention is read in conjunction with the accompanying drawings.

### BRIEF DESCRIPTION OF THE DRAWINGS

FIGS. 1A-1B show the amino acid sequence of IFNτ (FIG. 1A) and its tertiary structure (FIG. 1B);

FIG. 2 is a ultra violet (UV) circular dichroism (CD) spectrum of IFNτ showing the mean residue ellipticity as a function of wavelength, in nm;

FIG. 3 shows the fluorescence spectrum of IFNτ as signal as a function of wavelength, in nm;

FIG. 4 is a near UV CD spectrum for IFNτ showing the mean residue ellipticity as a function of wavelength, in nm;

FIG. 5 is the second derivative UV spectrum of IFNτ showing the second derivative of absorbance (ΔA²/Δλ²) as a function of wavelength, in nm;

FIG. 6 is a near UV CD spectra of three samples of IFNτ at concentrations of 0.5 mg/mL, 10 mg/mL, and 20 mg/mL in 20 mM histidine buffer, pH 7;

FIG. 7 shows the unfolding IFNτ in the presence of guanidinium hydrochloride, as a plot of mean residue ellipticity at 222 nm as a function of guanidinium concentration in moles/liter (M/L);

FIG. 8 shows far UV CD spectra for IFNτ taken at pH values of 3.5, 5.5, 7.4, and 8.5;

FIGS. 9A-9C are near UV CD spectra of IFNτ in phosphate buffer (FIG. 9A), in Tris buffer (FIG. 9B), and in histidine buffer (FIG. 9C) at time zero, prior to heating to 50° C. (solid line) and at 1 hour (triangles) and 3 hours (open circles) after heating;

FIGS. 10A-10C shows the results of dynamic light scattering analysis, where the percent mass as a function of hydrodynamic radius of the particle, in nm, for IFNτ formulated in phosphate buffer (FIG. 10A), Tris buffer (FIG. 10B), and histidine buffer (FIG. 10C). Samples were taken after formulation preparation at room temperature (time zero, open circles) and after heating to 50° C. for one hour (open triangles) and for three hours (filled squares);

FIGS. 11A-11B are fluorescence emission scans at 295 nm of IFNτ in histidine buffer, at histidine concentrations of 0 mM (solid line), 5 mM (open circles), 10 mM (filled squares), 15 mM (filled circles), 20 mM (filled triangles), 50 mM (open triangles), and 100 mM (filled bowties) (FIG. 11A) and a plot of the maximum fluorescence intensity from the data in FIG. 11A plotted against histidine concentration (FIG. 11B);

FIG. 12 is a plot of aggregation index (A.I.) as a function of NaCl concentration and pH for a solution of IFNτ heated to 50° C. for three hours;

FIG. 13 is a plot of percent of IFNτ, as determined by RP-HPLC, as a function of NaCl concentration and pH for a solution of IFNτ heated to 50° C. for three hours;

FIGS. 14A-14B are plots of mean residue ellipticity, determined by UV CD, as a function of wavelength, in nm, for solutions of IFNτ in propylene glycol (open circles, FIG. 14A) and in glycerol (open circles FIG. 14B), and for IFNτ in propylene glycol diluted 10-fold with Tris buffer (filled squares, FIG. 14A) and in glycerol diluted 10-fold with Tris buffer (filled squares, FIG. 14B), and for aqueous IFNτ (solid line in both figures); and

FIG. 15 is a plot of mean residue ellipticity, determined by UV CD, as a function of wavelength, in nm, for solutions of IFNτ in a simple syrup (open circles) and for an IFNτ control (solid line).

### BRIEF DESCRIPTION OF THE SEQUENCES

SEQ ID NO:1 corresponds to an amino acid sequence of mature ovine interferon-τ (OvlFNτ; oTP-1; GenBank Accession No. Y00287; PID g1358).

SEQ ID NO:2 corresponds to an amino acid sequence of mature ovine IFNτ, where the amino acid residues at positions 5 and 6 of the sequence are modified relative to the sequence of SEQ ID NO:1.

SEQ ID NO:3 corresponds to the amino acid sequence of fragment 1-37 of SEQ ID NO:1.

SEQ ID NO:4 corresponds to the amino acid sequence of fragment 1-28 of SEQ ID NO:1.

SEQ ID NO:5 corresponds to the amino acid sequence of fragment 2-27 of SEQ ID NO:1.

SEQ ID NO:6 corresponds to the amino acid sequence of fragment 1-37 of SEQ ID NO:2.

SEQ ID NO:7 corresponds to the amino acid sequence of fragment 1-28 of SEQ ID NO:2.

SEQ ID NO:8 corresponds to the amino acid sequence of mature human interferon-α (IFNτ-d; GenBank Accession No. J00210, PID g386796).

SEQ ID NO:9 is a hybrid interferon-τ/interferon-α protein, where residues 1-37 are interferon-τ residues (SEQ ID NO:6) and the remaining residues are interferon-α.

SEQ ID NO:10 is a hybrid interferon-τ/interferon-α protein, where residues 1-28 are interferon-τ residues (SEQ ID NO:7) and the remaining residues are interferon-α.

SEQ ID NO:11 is the amino acid sequence of an interferon-α analog having specific N-terminal residues replaced with interferon-τresidues.

SEQ ID NO:12 is the amino acid sequence of an interferon-α analog having specific N-terminal residues replaced with interferon-τ residues.

SEQ ID NO:13 is the amino acid sequence of an interferon-α analog having specific N-terminal residues replaced with interferon-τ residues.

SEQ ID NO:14 is the amino acid sequence of an interferon-α analog having specific N-terminal residues replaced with interferon-τ residues.

SEQ ID NO:15 is the amino acid sequence of an interferon-α analog having specific N-terminal residues replaced with interferon-τ residues.

SEQ ID NO:16 is the amino acid sequence of an interferon-α analog having specific N-terminal residues replaced with interferon-τ residues.

SEQ ID NO:17 is the amino acid sequence of an interferon-α analog having specific N-terminal residues replaced with interferon-τ residues.

SEQ ID NO:18 is the amino acid sequence of an interferon-α analog having specific N-terminal residues replaced with interferon-τ residues.

### DETAILED DESCRIPTION OF THE INVENTION

I. Definitions

The terms “protein” and “polypolypeptide” are used interchangably and refer to a polymer of amino acids and do not refer to a specific length of a polymer of amino acids. Thus, for example, the terms peptide, oligopolypeptide, and enzyme are intended to be encompassed by the term protein or polypolypeptide. This term also includes post-expression modifications of the polypolypeptide, for example, glycosylations, acetylations, phosphorylations, and the like.

“Interferon-tau”, abbreviated as IFNτ or interferon-τ, refers to any one of a family of interferon proteins having at least one characteristic from each of the following two groups of characteristics: (i) (a) anti-luteolytic properties, (b) anti-viral properties, (c) anti-cellular proliferation properties; and (ii) about 45 to 68% amino acid homology with α-Interferons and greater than 70% amino acid homology to known IFNτ sequences (e.g., Ott, et al., J. Interferon Res., 11:357 (1991); Helmer, et al., J. Reprod. Fert., 79:83(1987); Imakawa, et al., Mol. Endocrinol, 3:127 (1989); Whaley, et al., J. Biol. Chem., 269:10846 (1994); Bazer, et al., WO 94/10313 (1994)). Amino acid homology can be determined using, for example, the LALIGN program with default parameters. This program is found in the FASTA version 1.7 suite of sequence comparison programs (Pearson and Lipman, PNAS, 85:2444 (1988); Pearson, Methods in Enzymology, 183:63 (1990); program available from William R. Pearson, Department of Biological Chemistry, Box 440, Jordan Hall, Charlottesville, Va.). IFNτ can be obtained from ruminant species, including but not limited to cows, sheep, ox, horses, and deer.

“Interferon-alpha”, abbreviated IFNα or interferon-α, refers to any one of a family of interferon proteins having greater than 70%, or preferably greater than about 80%, or more preferably greater than about 90% amino acid homology to the mature IFNα protein sequence presented as SEQ ID NO:8.

II. Oral Formulation

In one aspect, the invention includes a composition for oral administration of a protein. The composition is comprised of the protein and a species effective to stabilize the protein. The stabilizing species in the working examples, described below, is a buffer. Selection of the buffer is made by evaluating the interaction between the buffer and the protein to determine the ability of a particular buffer to stabilize a given protein in a form that retains the activity of the protein upon oral administration, as will be described.

A. Interferon-τ as an Exemplary Protein

In studies performed in support of the invention, interferon-tau (IFNτ) was used as a model or exemplary protein for development of a suitable formulation for oral administration of any protein or polypeptide, more particularly for any cytokine, and more specifically for any interferon.

IFNτ is a type-1 interferon that is produced by the trophoblast of ruminant species, primarily ruminant ungulates. During pregnancy in ruminants, IFNτ acts a pregnancy signal, as it is secreted by the primitive trophoblast. Structurally, it displays a 4-helix bundle motif, typical of other type-1 interferons. IFNτ has antiproliferative, autoimmune, and antiviral properties (see, for example, U.S. Pat. Nos. 5,906,816; 5,942,223; 6,372,206; 5,958,402; WO 94/10301) and thus has potential as a therapeutic agent.

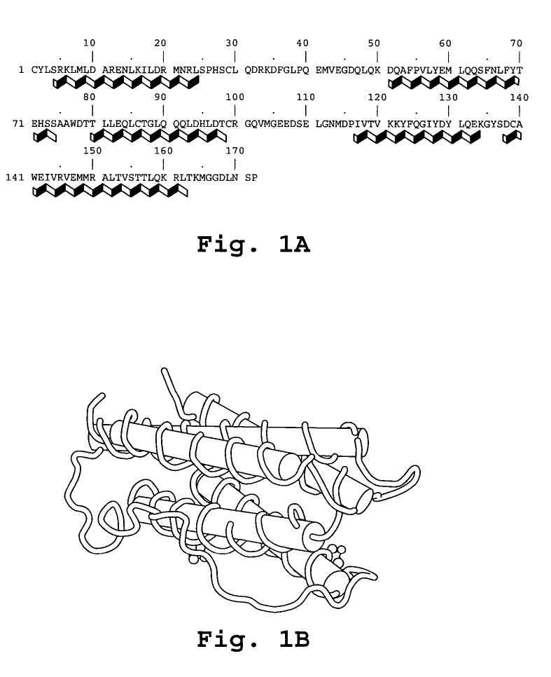

The 172 amino acid sequence of ovine-IFNτ is set forth, for example, in U.S. Pat. No. 5,958,402, and its homologous bovine-IFNτ sequence is described, for example, in Helmer et al., J. Reprod. Fert., 79:83-91 (1987) and Imakawa, K. et al., Mol. Endocrinol., 3:127 (1989). The sequences of ovine-IFNτ and bovine-IFNτ from these references are hereby incorporated by reference. The amino acid sequence of ovine IFNτ is shown in FIG. 1A and identified herein as SEQ ID NO:1, and analysis of the sequence predicts four major helices present in IFNτ, as illustrated in FIG. 1B, and this has been confirmed by x-ray crystallography. The four helices assemble to form a four-helix bundle motif (Li, J.; et al., J. Biol. Chem. 40:7, 24826-24833 (1994); Radharkrishnan, R. et al., J. Mol. Biol., 286:151-162 (1999); Senda, T. et al., J. of Interferon and Cytokine Research, 15:1053-1060 (1995)). A second amino acid sequence of ovine IFNτ is identified herein as SEQ ID NO:2.

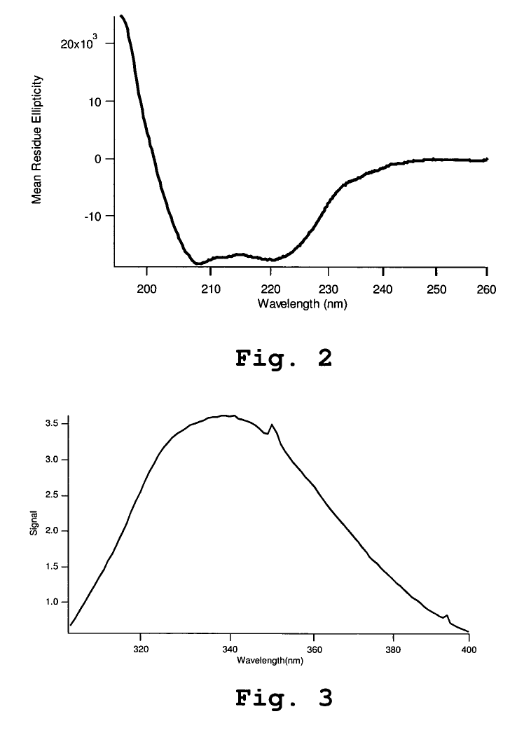

The secondary structure of IFNτ was investigated by the use of far UV circular dichroism (CD). FIG. 2 shows the far UV CD spectrum for IFNτ. The negative bands near 222 and 208 nm are indicative of a large amount of α-helical content. Analysis of the spectrum yielded an estimate of the α-helical content of approximately 53%. This is consistent with the published crystal structure of IFNτ where 58% of the residues are in an alpha-helical conformation (Radharkrishnan, R. et al., J. Mol. Biol, 286:151-162 (1999)).

The tertiary structure of IFNτ was investigated using fluorescence spectroscopy. FIG. 3 shows the fluorescence spectrum of IFNτ where the signal as a function of wavelength is shown graphically. The intrinsic fluorescence spectrum of IFNτ exhibits a maximum at 338 nm (λₑₓ at 295 nm), indicating that at least one of the two tryptophan residues (at positions 77 and 141) are relatively protected from the solvent. The crystal structure of IFNτ shows that Trp⁷⁷ is packed into the hydrophobic core of the molecule between helices A, C, and D (Radharkrishnan, R. et al., J. Mol. Biol., 286:151-162 (1999)). The hydrophobic pocket is formed by amino acid residues Cys¹, Leu⁷, Phe⁵⁴, Leu⁵⁷, Leu⁹⁶, Cys⁹⁹, and Leu¹⁶². In other type 1 interferons, Trp⁷⁷ packs into the hydrophobic core of the molecule near Trp¹⁴¹ (on helix E) packed on the interior of the B-D helix interface.

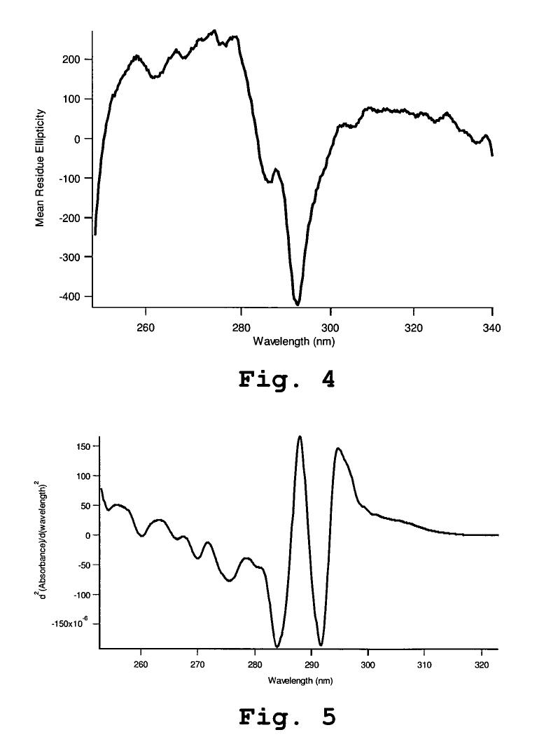

The tertiary structure of IFNτ was also examined using near UV CD spectroscopy and the results are shown in FIG. 4. The near UV CD spectrum, which arises from signals from the aromatic side chains, indicates the presence of well-organized tertiary structure. There is a strong negative band near 292 nm, arising from a vibronic component of the L_{b} band of Trp. This observation is consistent with one or both of the Trp residues residing in a well-defined environment. The presence of organized tertiary structure is also detected in the second derivative UV spectrum of IFNτ, as shown in FIG. 5. There are two strong negative bands, one at about 292 nm and the other at about 284 nm. The first is due to vibronic fine structure of Trp residues, much like the features seen in the near UV CD spectrum. The second negative peak arises from the Tyr residues. The r value of 1.00 is consistent with the presence of a globular structure, with both Tyr and Trp residues being at least partially solvent protected.

B. Structure and Stability Studies

B.1. Concentration Effects on the Structure and Stability of IFNτ. The association state of proteins can change with increasing concentrations. In order to ascertain the effect of high protein concentrations on the structure and stability of IFNτ, samples of varying concentrations in a buffer were prepared and analyzed by near UV CD. Samples of IFNτ at concentrations of 0.5 mg/mL, 10 mg/mL, and 20 mg/mL in 20 mM histidine buffer, pH 7 were prepared and analyzed. FIG. 6 shows the near UV CD spectra of these three samples. It is evident from the spectra that the spectra are slightly perturbed at higher concentrations. This may be due, in part, from small changes in the tertiary structure of the protein or from association between protein molecules at higher concentration. The protein is still mostly native-like at concentrations up to 20 mg/mL in aqueous solution, and the studies suggest that IFNτ can be concentrated to approximately 50 mg/mL without significant effects on conformation.

B.2. Thermal Unfolding Studies. Proteins can be denatured by various stresses. The two most common are addition of a chaotrope, such as GnHCl, or by an increase in temperature. Measurements of thermal unfolding provoked by a selected stress provide qualitative comparisons of the relative stability of different formulations, and indicate the actual thermodynamic conformational stability of the protein. In support of the present invention, thermal unfolding studies were performed by monitoring the CD spectrum at 222 nm, the wavelength most sensitive to a change in α-helical content. An unfolding curve was determined for IFNτ at a concentration of 0.05 mg/mL in 20 mM tris, 100 mM NaCl, pH 7.0. The Tₘ value (the midpoint of the unfolding transition) was determined to be 69° C., with an apparent onset of approximately 55° C. The thermal denaturation of IFNτ under these conditions was mostly irreversible, therefore, thermodynamic parameters were not calculated from this data set. The data indicates that IFNτ is relatively thermally stable, with no appreciable denaturation (less than 2%) occurring below 50-55° C.

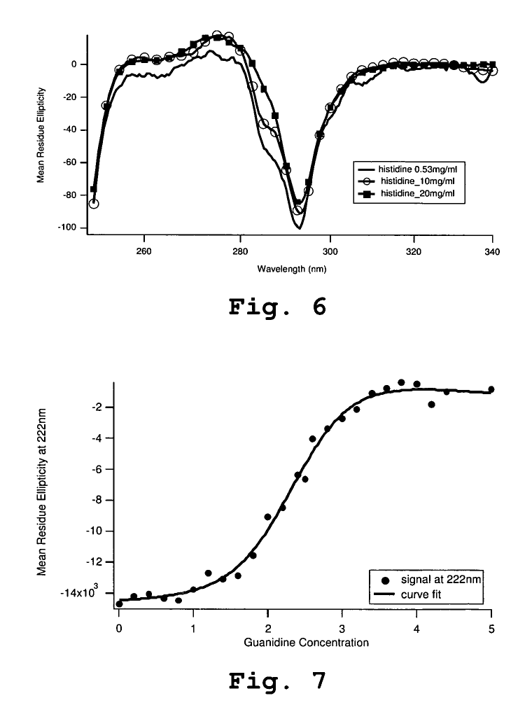

B.3. Guanidinium Hydrochloride (GnHCl) Unfolding Studies. An estimate of the thermodynamic stability of ovine IFNτ was attempted by measuring the degree of unfolding as a function of GnHCl at 25° C. The results are shown in FIG. 7 as mean residue ellipticity at 222 nm as a function of guanidinium concentration in moles per liter (M/L). A cooperative unfolding transition was observed, with a midpoint near 2.5 M GnHCl. Extrapolation to zero GnHCl concentration gave an estimate of the free energy of unfolding of 4.2 kcal/mole. Curve fitting provided an estimate of 3.8 kcal/mole. Therefore, it appears that at 25° C., the protein displays a free energy of unfolding of approximately 4 Kcal/mole.

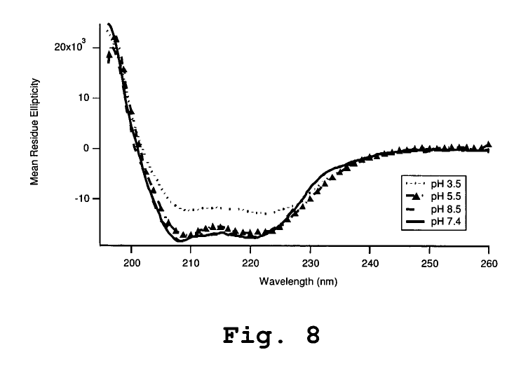

B.4. pH Effects on the Structure of IFNτ. The pH of a protein can have a large affect on the structure as well as the protein's stability (Yang, A. et al., J. Mol Biol., 237:602-614 (1994); Lakemond, C. M. M. et al., Journal of Agricultural And Food Chem., 48:(6):1985-1990 (2000)). Therefore, in another study performed in support of the invention, the secondary structure of IFNτ as a function of pH was determined using far UV CD. In this study, solutions of IFNτ at pH values of 3.5, 5.5, 7.4, and 8.5 were prepared and analyzed by far UV CD. The results are shown in FIG. 8. Samples at pH 3.5 displayed a modest loss of α-helical content (˜45%), when compared to approximately 53% for the native protein at pH 7.4. At the other two pH values (5.5 and 8.5), the helix content was near that of the native protein at pH 7.4. At even lower pH (pH 2), the α-helical content of IFNτ appears to be near 45% (data not shown). In general, it appears that incubation of IFNτ below pH 4 results in slightly perturbed secondary structure. Despite exposure to low pH, IFNτ does not appear to unfold under highly acidic conditions, as seen in proteins such as apomyoglobin or horse myoglobin (Yang, A. et al., J. Mol. Biol., 237:602-614 (1994); Chi, Z. et al., Biochemistry, 37:2865-2872 (1998)).

As for the tertiary structure, the r value (defines as the a/b ratio), derived from the second derivative UV spectrum, indicates that the microenvironments of the aromatic side chains in IFNτ are more solvent exposed, perhaps due to a slight rearrangement of the globular struture (Senda, T. et al., J. of Interferon and Cytokine Research, 15:1053-1060 (1995); Servillo, L. et al., Anal. Biochemistry, 126:251-257 (1982); Kornblatt, J. A. et al., Biochemistry, 34:1218-1223 (1995)).

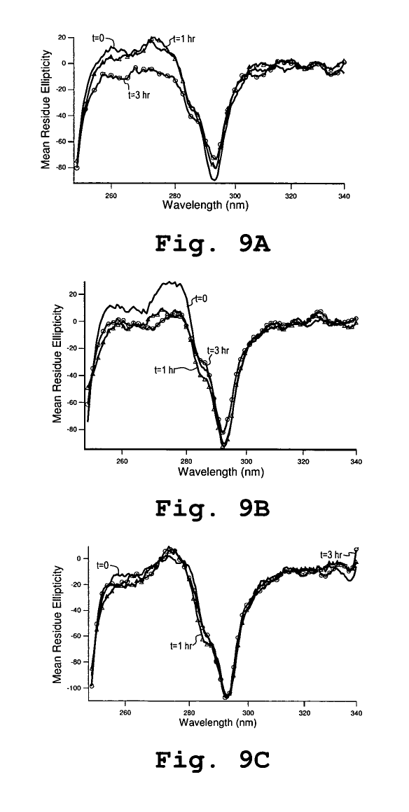

B.5. Effect of Buffer Species on the Stability and Structure of IFNτ. Based on the results in FIG. 8 showing that solutions with a pH value around 7 produce a native-like structure, another study was performed to examine the effect of various buffer species on the stability and structure of IFNτ. In this study, three different buffers capable of maintaining the pH of the solution at 7 were evaluated. The buffers were Tris, phosphate, and histidine. The buffer concentration was fixed at 20 mM and the salt concentration was held constant at 100 mM. Samples were heated at 50° C. and aliquots were taken at one and three hours and compared to initial samples (t=0 hours) by near UV CD. The results are shown in FIGS. 9A-9C.

As seen in FIG. 9A, the near UV CD spectra of the IFNτ in phosphate buffer displayed a marked loss in tertiary structure upon heating. Similarly, as seen in FIG. 9B, solutions of IFNτ in Tris buffer showed a loss of tertiary structure. However, IFNτ formulated in a solution containing histidine as the buffer had little if any loss of tertiary structure in the near UV CD spectra after heating. Thus, histidine enhances the thermal stability of IFNτ relative to either phosphate or Tris buffers.

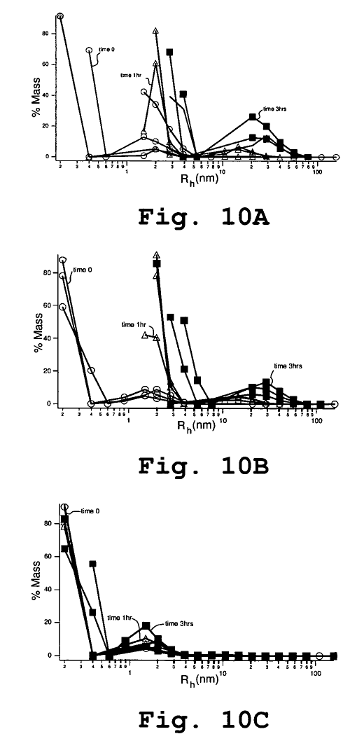

in another study, the thermal stability of IFNτ in phosphate, Tris, and histidine buffer solutions with a pH value of about 7 was monitored using dynamic light scattering (DLS). After taking an initial sample for measurement, the IFNτ solutions were heated at 50° C. Samples were taken 1 hour and 3 hours after heating for measurement by DLS, where an increase in the hydrodynamic radius of the particle is evidence of protein association or aggregation. The results are shown in FIGS. 10A-10C.

FIG. 10A shows the DLS measurements of IFNτ formulated in phosphate buffer after formulation preparation at room temperature (time zero, open circles) and after heating to 50° C. for one hour (open triangles) and for three hours (filled squares). Formation of large aggregates is apparent as evidenced by the increase in average hydrodynamic radius from 20 nm at 1 hour to 40 nm at 3 hours. FIG. 10B shows a similar profile for IFNτ formulated in Tris buffer, although the intensity from each aggregate ensemble is less than with phosphate. IFNτ formulated in histidine buffer, seen in FIG. 10C, shows a low intensity of aggregated species smaller aggregates. Thus, IFNτ formulated in a histidine-containing solution exhibits significantly lower amounts of soluble aggregates.

Formation of soluble aggregates is easily monitored by size-exclusion chromatography-high pressure liquid chromatography (SEC-HPLC), where the amount of aggregate can be more accurately quantitated. For SEC-HPLC studies performed in support of the invention, samples of IFNτ (1.0 mg/mL) in phosphate buffer, Tris buffer, and histidine buffer were heated to 60° C. for up to two hours and analyzed by SEC-HPLC. The results are shown in Table 1. IFNτ formulated in phosphate and Tris containing solutions displayed significant aggregation, even within the first 30 minutes. In contrast, little aggregation was observed in the histidine-containing samples. All of these studies indicate that there is a significant enhancement of the thermal stability of IFNτ in the presence of histidine buffer, relative to the protein formulated in phosphate or Tris buffers.

TABLE 1
Percent Soluble Aggregate formation in IFNτ (1.0 mg/mL) in the
presence of different buffer species during thermal treatment
at 50° C.

| Formulation | 0 min | 30 min | 60 min | 90 min | 120 min |
|---|---|---|---|---|---|
| 20 mM Tris/100 mM NaCl | 0.44 | 2.6 | 6.2 | 9.7 | 11.95 |
| 20 mM phosphate/100 mM | 0.5 | 4.4 | n.t | 16.3 | 18.4 |
| NaCl | | | | | |
| 20 mM histidine/100 mM | 0 | 1.0 | 1.8 | n.t. | n.t. |
| NaCl | | | | | |
| n.t. = not tested. | | | | | |

B.6. Thermally-induced Aggregation Kinetic Studies. Histidine-induced stabilization of IFNτ was further studied via kinetic studies of thermally-induced aggregation. Proteins, such as interferon-γ and interleukin-1 receptor antagonist, aggregated upon heating following first-order kinetics (Kendrick, B. S. et al., Proc. Natl. Acad. Sci. USA, 94:11917-11922 (1997); Kendrick, B. S. et al., Proc. Natl. Acad. Sci. USA, 95:14142-14146 (1998)). Examination of the aggregation rate as a function of protein concentration indicated that IFNτ displayed the same behavior (data not shown).

In each of the previous cases, the rate-limiting step was a conformational change from the compact native state to a slightly expanded aggregation-competent conformation (Kendrick, B. S. et al., Proc. Natl. Acad. Sci. USA, 95:14142-14146 (1998)). Moreover, it has been demonstrated that addition of preferentially excluded solutes, such as sucrose (Lee, J. C. et al., J. Biol. Chem., 256:7193-7201 (1981)), increase the chemical potential of the protein, which is directly proportional to the surface area of protein exposed to solvent. This, by the LeChatelier Principle, will shift the equilibrium toward to more compact native state (Wyman, J., Adv. Protein Chem., 19:223-286 (1964)), thereby slowing the protein aggregation rates. The degree of expansion can be determined by measuring the rate of aggregation as a function of sucrose concentration.

$$\frac{\partial ln v}{\partial σ} = \frac{Δ s}{RT} \qquad ( 1 )$$

In equation 1, v is the apparent rate of aggregation as determined by SEC-HPLC, σ is the surface tension of the solution, Δs/RT is the surface area change of the protein going from N to N*, where N is the native protein and N* is the expanded aggregation-competent conformation.

A study was performed for IFNτ in the presence phosphate buffer, Tris buffer, and histidine, with the buffer concentration at 20 M, the salt concentration at 100 mM, and the protein concentration at 1 mg/mL. The change in surface area, Δs, was calculated from the slope of the plot of in v (the apparent rate constant) vs. the surface tension of the solution. The results are shown in Table 2.

TABLE 2

| | Δs/RT | | % |
|---|---|---|---|
| | (m = meters) | Δs/molecule | surface |
| | (N = | (nm² = square | area |
| Formulation | Newtons) | nanometers) | change |
| 20 mM Tris/100 mM NaCl | 502.1 m/N | 2.25 nm²/molecule | 5.08% |
| 20 mM phosphate/100 mM | 479.9 m/N | 2.15 nm²/molecule | 4.87% |
| NaCl | | | |
| 20 mM histidine/100 mM | 316.1 m/N | 1.41 nm²/molecule | 3.25% |
| NaCl | | | |

For histidine-containing samples, the value of Δs was found to be 1.41 nm²/molecule. As native IFNτ has an overall surface area of 42 nm² (determined from the crystal structure), this represents an increase of 3.8% in total surface area for the aggregation-competent state. Likewise, the surface area change in phosphate and Tris are 2.15 nm²/molecule and 2.25 nm²/molecule, respectively (see Table 2). These findings indicate that the less stable formulations have a more expanded aggregation-competent state. The Δs suggests that the expanded state, N*, is more native-like in the presence of histidine than it is in the presence of the other buffers species.

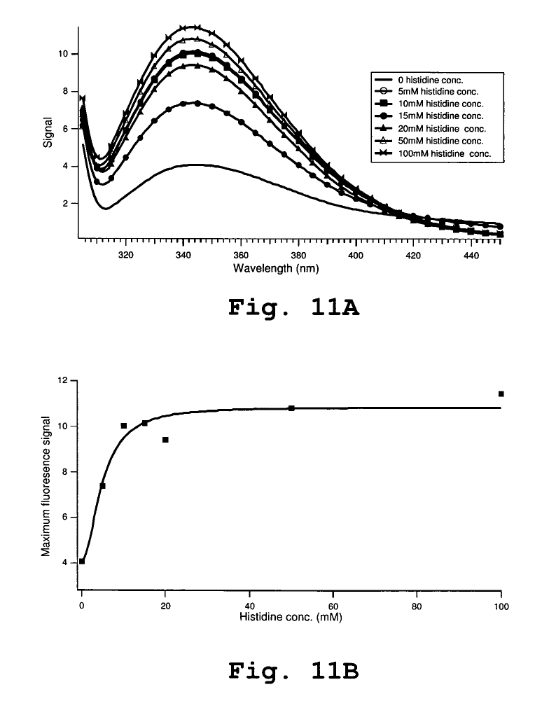

B.7. Histidine-Binding Studies. In another study performed in support of the invention, binding of histidine was examined by monitoring the intrinsic tryptophan fluorescence of IFNτ in the presence of various amounts of histidine. Fluorescence emission scans of each sample were collected by exciting at 295 nm, which is selective for tryptophan residues. The results are shown in FIGS. 11A-11B, where the signal of IFNτ tryptophan in histidine concentrations of 0 mM (solid line), 5 mM (open circles), 10 mM (filled squares), 15 mM (filled circles), 20 mM (filled triangles), 50 mM (open triangles), and 100 mM (filled bowties) histidine was measured as a function of wavelength. FIG. 11A shows the fluorescence intensity increases with increasing histidine concentrations. A plot of the maximum fluorescence intensity vs. histidine concentration, FIG. 11B provides an estimation of the binding constant. These studies show that histidine stabilizes the protein by binding, to stabilize the protein's globular structure for activity retention.

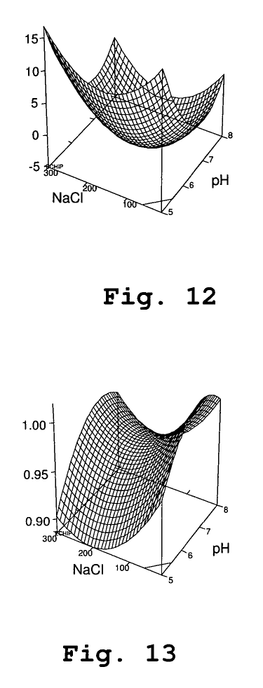

B.8. Accelerated Storage Studies. Ionic strength can affect the solution properties by modulating the protein's stability and solubility (Arakawa, T., et al., Biochemistry, 21:6545-6552 (1982)). A means of adjusting the ionic strength of a solution is by the addition of sodium chloride (NaCl). In another study, the effect of NaCl concentration on stability of IFNτ was evaluated. In this study, samples of IFNτ were stored at 50° C. for three hours at various pH levels and NaCl concentrations. The samples by were analyzed by aggregation index (A.I.), reversed phase HPLC (RP-HPLC) and anti-viral activity, to determine the overall stability of IFNτ as a function of solution conditions. The results are shown in FIG. 12 and FIG. 13.

FIG. 12 shows the aggregation index (A.I.) appears to be minimal at a pH of 7 and with NaCl concentrations between 100 mM and 200 mM. Recovery of initial IFNτ, as determined by RP-HPLC, appears to be maximal at pH 7 and at low NaCl concentrations, as seen in FIG. 13. The results from the anti-viral activity assay indicate that the specific activity appears to be maximal between pH 7 and 8 and at low NaCl concentrations (data not shown). Together, these data indicate that a pH value of around 7.0, more generally of between about 6.8-7.2, and still more generally of between about 6.5-7.5 achieve IFNτ stability at elevated temperatures. A NaCl concentration of less than about 140 mM, more preferably of less than about 125 mM, and still more preferably of less than about 100 mM results in a stable IFNτ formulation.

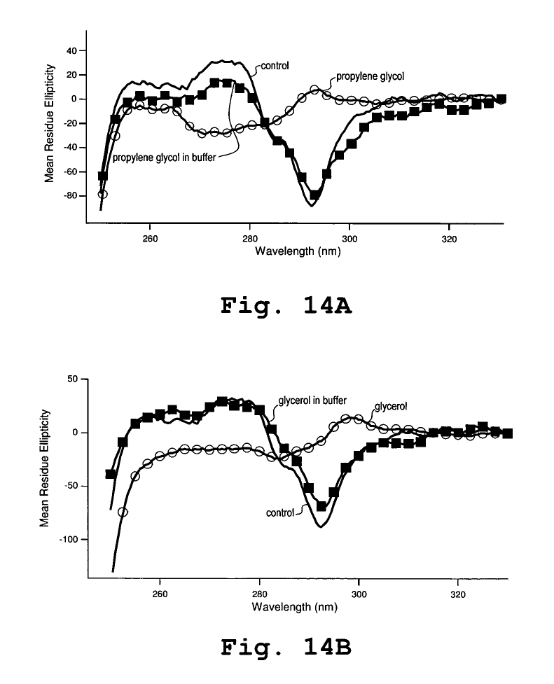

B.9. Effect of Nonageuous Solvents on the Structure and Stability of IFNτ. As noted above, oral delivery of proteins and polypeptides is of considerable interest. Thus, in another study, formulations compatible with gelatin capsules were examined. Placement of a liquid formulation inside a gelatin capsule generally requires the water content to be less than about 10%, frequently less than about 5%. To achieve a low water concentration, a nonaqueous solvent, such glycerol or propylene glycol, is employed. The effect of these solvents in the structure and stability of IFNτ was examined by UV CD, as shown in FIGS. 14A-14B.

FIG. 14A shows the mean residue ellipticity as a function of wavelength, in nm, for solutions of IFNτ in propylene glycol (open circles), in propylene glycol diluted 10-fold with Tris buffer (filled squares), and for aqueous IFNτ (solid line). FIG. 14B is a similar plot for IFNτ in glycol (open circles), in glycerol diluted 10-fold with Tris (filled squares) and for aqueous IFNτ. For both propylene glycol and glycerol, it is apparent that there are severe structural perturbations of the tertiary structure in the presence of a concentrated organic solvent. However, when the organic solvent is diluted ten-fold with Tris buffer, the tertiary structure appears to return to an almost native-like structure for both the propylene glycol and glycerol formulations.

The perturbation of the secondary structure of IFNτ in the presence of propylene glycol and glycerol was investigated using far UV CD spectroscopy. There are some changes in the secondary structure composition in the presence of high concentrations of the organic solvent, but the changes were modest. Again, upon a ten-fold dilution of either propylene glycol or glycerol into aqueous buffer, the secondary structure of IFNτ is nearly identical to the native protein. This suggests that any structural change that occurs in these formulations is reversible.

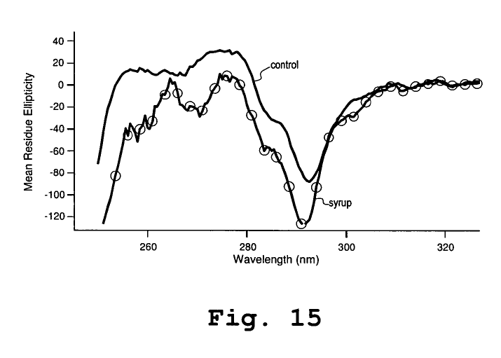

Another suitable oral formulation for proteins and polypeptides is an ingestible syrup or elixir. IFNτ was formulated into a USP sucrose syrup and analyzed by near UV CD. The spectrum is shown in FIG. 15 and shows that the tryptophan features become more intense in the near UV CD spectrum. This suggests that the environments of the tryptophan residues become better defined from an increase in ordering of the tertiary structure.

Based on the foregoing, it can be appreciated that similar evaluations of a protein's response to a selected buffer can be evaluated to ascertain the buffer capable of stabilizing the protein by retaining the protein's tertiary structure to ensure activity of the protein after formulation and upon oral administration. Thus, the invention contemplates a method for selecting an oral formulation for a protein that achieves protein stabilization for biological activity upon in vivo administration. The method includes (I) selecting a protein for formulation into a dosage form; (ii) preparing solutions of the selected protein in selected buffers at selected pH levels; and) measuring the effect of the buffer on the structure and/or stability of the protein to identify one or more buffers that result in a relatively small change in the protein's tertiary structure. Retention of tertiary structure in directly related to retention of biological activity for most proteins.

More generally, the invention contemplates a method of preparing a protein for in vivo, and in particular oral, administration. Oral administration of proteins is technically challenging because of the enzymatic degradation of the protein in stomach and intestinal tract. Identification of a formulation where the protein is stabilized in its active form is desirable. As evidenced by the studies described above, an oral preparation that includes a species effective to stabilize a protein in its active form by binding interaction with the protein can be identified for any given protein. In the case of interferon-τ, a histidine buffer effectively stabilized the protein as evidenced by retention of the α-helical tertiary structure when subjected to conditions that would normally cause a reduction of tertiary structure. In particular, a histidine buffer based on L-histidine (Sigma, USP grade) in water at a histidine concentration of 20 mM, pH 7.0-7.2, was shown to stabilize interferon-τ. The pH of the histidine buffer after initial preparation was about 7.4; the pH was reduced with 1 N HCl to about 7.0-7.2. More generally, formulation of an oral dosage form containing a type I interferon and L-histidine buffer at a concentration of between about 10-80 mM, more prefereably between 10-30 mM, at pH between 6-8, more preferably at pH between 6.9-7.2, is contemplated.

It will be appreciated that the findings described herein are suitable for any protein or peptide, including fusion proteins and hybrid proteins. In particular, the advantages of a liquid preparation comprised of histidine on hybrid proteins based on interferon-τ are contemplated. Interferon-τ hybrid proteins have been described, for example, U.S. Pat. Nos. 5,939,286 and 6,174,996, where fusion peptides comprised of interferon-τ and a non-tau interferon type I protein were disclosed. The disclosure of these patents, including the sequences of the various fusion proteins, is incorporated by reference herein. In particular, hybrid interferon fusion proteins having an N-terminal region of interferon-τ and a C-terminal region of another type I interferon are preferred. The N-terminal region of interferon-τ is responsible for the reduced cytotoxicity associated with interferon-τ, relative to other type I interferons, such as interferon-α. Typically, the first 8-37 amino acid residues of a non-tau interferon are replaced with an identical number of residues from the first 8-37 residues of interferon-τ. One exemplary hybrid protein is formed by replacing the N-terminal region of a non-tau type I interferon by an N-terminal region of interferon-τ corresponding to a sequence spanning residues 1 to 37 of interferon-τ. Residues 1-37 of ovine interferon-τ are presented herein as SEQ ID NO:3 and SEQ ID NO:6 Other exemplary N-terminal sequences of interferon-τ include residues 1-28 (SEQ ID NO:4 and SEQ ID NO:7) and residues 2-27 (SEQ ID NO:5). Exemplary hybrid protein sequences having a defined number of residues from the N-terminal region of interferon-τ and the remaining residues of a non-tau, type I interferon are shown in SEQ ID NO:9 and SEQ ID NO:10.

In another embodiment, the protein included in the liquid formulation described herein is a modified non-tau, type I interferon protein, such as those described in U.S. Pat. No. 6,204,022, incorporated by reference herein. In such modified interferon proteins certain selected amino acid residues in the N-terminal region of the non-tau, type I interferon are substituted with the interferon-τ residue at the corresponding position. For example, selected amino acid residues in interferon-α, identified herein as SEQ ID NO:8, are substituted with one or more amino acid residues of interferon-τ. In particular, introducing interferon-τ amino acid residues at one or more of positions 19, 20, 22, 24, and 27 in mature interferon-α provides a modified interferon-α protein with reduced cytotoxicity. An exemplary modified interferon-α protein is presented as SEQ ID NO:11, where native interferon-α is modified at amino acid residue positions 19, 20, 22, 24, and 27 with the corresponding residue of interferon-τ. It will be appreciated that one or more of amino acid residues 19, 20, 22, 24, and 27 can be substituted and that residues other than these specific residues can be additionally modified. Additional exemplary modified interferon-α proteins are presented herein as SEQ ID NO:12 through SEQ ID NO:18. In this embodiment, amino acid residue 19 of interferon-α is substituted with Asp¹⁹ of mature ovine-interferon-τ, or with a same-class residue Asn, Gln, or Glu; amino acid 20 of mature interferon-α may be substituted with Arg²⁰ of mature ovine-interferon-τ or with a same-class residue His or Lys; amino acid 22 of mature interferon-α may be substituted with Asn²² of mature ovine-interferon-τ or with a same-class residue Asp, Gln, or Glu; amino acid 24 of mature interferon-α may be substituted with Leu²⁴ of mature ovine-interferon-τ or with a same-class residue Val or Met; and amino acid 27 of mature interferon-α may be substituted with His²⁷ of ovine-interferon-τ or with a same-class residue Arg or Lys. In all these modified interferon-α proteins, a modified protein where only position 22 in the native interferon-α amino acid sequence is substituted with Ser, Thr, Asn, Gin, or Gly is excluded. Modified interferon proteins constructed according to the substitutions described above exhibit a reduced toxicity relative to native human interferon-α with no significant alteration of desirable therapeutic properties.

From the foregoing, it can be seen how various objects and features of the invention are met. The solution behavior of IFNτ, as an exemplary protein, resulted in a determination of an appropriate buffer, ionic strength, and pH for an oral formulation. The conditions identified herein preserve the structure and ensure stability under accelerated storage conditions. Surprisingly, histidine by its binding to the protein provides a significant level of stabilization relative to other buffers. This effect is applicable to native proteins as well as fragments of proteins, hybrid proteins, and modified proteins.

### II. EXAMPLES

The following examples further illustrate the invention described herein and are in no way intended to limit the scope of the invention.

1. Materials

Three different lots of IFNτ (SEQ ID NO:1; SEQ ID NO:2) were provided by Pepgen Corporation, typically as frozen bulk at 1 mg/mL in pH 7.4, 20 mM Tris, 150 mM NaCl buffer.

2. Methods

RP-HPLC Method. Purity, identity, and concentration were assessed by RP-HPLC. The analysis was performed using a Vydec C4 column (S/N 570520-8-4) at a flow rate of 0.43 ml/min and a 10 μL sample injection volume. Solvent A was 0.1% trifluoroacetic acid (TFA) and solvent B was 0.1% TFA/99.9% acetonitrile (ACN). The column was equilibrated with a mixture of 90% A and 10% B. Upon sample injection, a thirty minute gradient up to 90% B/10% A was employed. This mixture was maintained for 35 minutes. Then, the solvent mixture was changed to 90% A/10% B and held there for 50 minutes. Under these conditions, the IFNτ peak eluted at approximately 22 minutes.

SEC-HPLC Method. Size exclusion was performed using TSD 9EL SUPER SW2000xI Tosohaas column (4.3 mm×30 cm, 5 um beads) using PBS as the mobile phase at a flow rate of 0.35 ml/min and an injection volume of 5 μL. The IFNτ peak eluted at 10 minutes with large aggregates eluting in the void volume at 6 minutes.

Electrophoresis Sizing Method. Sizing was performed under both reducing and non-reducing conditions using Protein Chips on an Agilent Bioanalyzer according to recommended protocols. Briefly, 4 μL of sample was mixed with 2 μL of denaturing buffer, samples were heated briefly (NMT 5 min) at 100° C. and diluted with the running buffer 15-fold. Samples (6 μL) were loaded on the Protein Chip and analyzed using the Bioanalyzer. The bands were calibrated using internal low MW and high MW markers. The samples were analyzed using the Bioanalyzer software. This technique provided semi-quantitive and qualitative information and amounts of relative % of the main IFNτ peak and aggregates.

Circular Dichroism Spectroscopy. Circular dichroism (CD) spectra were obtained on an AVIV model 62 DS spectropolarimeter (Lakewood, N.J.). Measurements of the samples were taken in a 0.5 cm pathlength quartz cell and placed in a thermostated cell holder. Concentrations were determined by UV/VIS spectrophotometry at 280 nm using an extinction coefficient of 0.95 (mL/mg*cm). For near UV CD spectra, data were collected at 0.5 nm intervals using a 1.0 nm bandwidth, with an averaging time of 5 seconds at each point. For far UV spectra, data were collected at 0.5 nm intervals utilizing a 1.5 nm bandwidth, with an averaging time of 5 seconds at each point. The appropriate buffer blank was collected and subtracted from each spectrum.

Thermal Melting Curves. Temperature melt curves were obtained by monitoring the CD signal of IFNτ samples at 222 nm on an Aviv model 62 DS spectropolarimeter (Lakewood, N.J.). Concentration determination was done on a Beckman DU-64 spectrophotometer using an extinction coefficient of 0.95 (mL/mg*cm). Each sample was loaded into a 0.1 cm pathlength quartz cell placed in a thermostated cell holder. Far UV CD wavelength data was collected prior to temperature treatment, at the warmest temperature, and after cooled to beginning temperature. Data was also collected during the temperature treatment by monitoring 222 nm, a band characteristic of alpha helix. All spectra were corrected and converted into mean residue ellipticity by the equation of [millidegrees*(mean residue weight/concentration(mg/mL)*pathlength(mm)].

Guanidine-hydrocholoride (GdnHCL) Denaturation. Following the changes in the CD signal at 222 nm enabled the observation of GdnHCL unfolding of IFNτ. GdnHCL concentrations were determined by refractive index measurements, as described elsewhere (Pace, C. N., Methods Enzymol., 131:266-280 (1986)), on a refractometer (Bausch & Lomb). The protein solutions were prepared and were equilibrated at room temperature overnight. The CD signal at 222 nm was obtained in a 0.1 cm pathlength quartz cell. The data were analyzed by both linear extrapolation and non-linear least squares fitting obtain the free energy of unfolding, ΔGᵤ, and the dependence of the free energy of unfolding on the denaturant concentration in the transition region, and the m value.

ΔG=ΔGᵤ−m[GdnHCL] (2)

where ΔGᵤ is the free energy of unfolding in the absence of GdnHCL and ΔG is the free energy of unfolding as a function of GdnHCl (Pace, C. N., Methods Enzymol., 131:266-280 (1986)).

Differential Scanning Calorimetry. Thermal denaturation was determined by differential scanning calorimetry using a Seiko DSC 6100 instrument. Denaturation profile was measured using a scan rate of 2° C./min from 0° C. to around 100° C. The onset temperature of denaturation, heat of denaturation (ΔH in mJ/mg), and the maximum melting temperature (Tₘ) was assigned as the temperature that the protein was totally unfolded. Sample size was 15 μL and all measurements were done utilizing silver sample pans that were sealed hermetically. The reference pan contained the corresponding buffer without protein.

Dynamic light scattering. The hydrodynamic radius of PG-301 was measured by use of dynamic light scattering. The dynamic light scattering measurements were made on a DynaPro-801 TC molecular sizing instrument (Protein Solutions, Charlottesville, Va.). Samples are drawn into a gas tight 250 μL syringe. The sample are filtered through a 0.02-0.2 μm Whatman filter as it is injected into the instrument. Sizes and distributions are calculated using the Dynamics software package (Protein Solutions).

Second Derivative Ultraviolet Spectroscopy. UV data were collected in a 1 cm pathlength quartz cell on a Hewlett Packard 8452A diode array spectrophotometer. The data was analyzed using Grams 386 software by use of the truncate, spline, and derivative functions to manipulate the data. The second derivative data were imported into a spreadsheet for data analysis. The a, b, and r values were calculated/taken from this second derivative data.

Aggregation Index Determination. Aggregation in the IFNτ samples were also assessed using an aggregation index (A.I.) value calculated from the spectroscopic values. A.I. values were calculated using 280 nm and 340 nm values using the following equation; A.I.=100×(A340)/(A280−A340). A.I. values below 10 represent solutions with insignificant amounts of insoluble aggregates and appear as clear and colorless solution.

Anti-viral Activity. Selected stability samples were analyzed for in-vitro anti-viral activity on MDBK cells as described by Rubinstein et al. (J. Virology, 37:755-758 (1981)). Samples were delivered as 50 μL aliquots stored at 2-8° C.

Although the invention has been described with respect to particular embodiments, it will be apparent to those skilled in the art that various changes and modifications can be made without departing from the invention.

It is claimed:

1. A composition for oral administration of an interferon, comprising
an interferon and a species effective to stabilize the interferon in an active form by interaction between the interferon and the species.

2. The composition of claim 1, wherein said species is a buffer.

3. The composition of claim 2, wherein said buffer is histidine.

4. The composition of claim 1, wherein the interferon is a type I interferon.

5. The composition of claim 4, wherein said interferon is selected from interferon-α, interferon-β, interferon-ω, and interferon-τ.

6. The composition of claim 1, wherein said protein is interferon-τ.

7. The composition of claim 1, wherein said protein is a hybrid protein comprising selected N-terminal residues of interferon-τ.

8. The composition of claim 7, wherein said hybrid protein is comprised of an N-terminal segment corresponding to amino acid residues 1 to about amino acid residue 28 of interferon-τ, and a C-terminal segment corresponding to about residue 29 to the C-terminal residue of interferon-α.

9. The composition of claim 7, wherein said hybrid protein is comprised of an N-terminal segment corresponding to amino acid residues 1 to about amino acid residue 37 of interferon-τ, and a C-terminal segment corresponding to about residue 38 to the C-terminal residue of interferon-α.

10. The composition of claim 7, wherein said hybrid protein is comprised of an N-terminal segment of amino acid residues 1-27 of interferon-α having modifications at one or more of positions 19, 20, 22, 24, and 27 and a C-terminal segment corresponding to residue 28 to the C-terminal residue of interferon-α.

11. The composition of claim 1, wherein said species is effective to bind with said interferon to stabilize its α-helical tertiary structure.

12. The composition of claim 1, wherein said composition has a pH value of between about 6-8.

13. The composition of claim 1, wherein said interferon is interferon-τ, said species is histidine at a concentration of between about 10 mM to about 80 mM.

14. The composition of claim 1, wherein said interferon is interferon-τ, said species is histidine at a concentration of about 20 mM.

15. A method of preparing a protein for oral administration, comprising
formulating said protein with a species effective to stabilize the protein in an active form by binding interaction between said protein and said species,
whereby said formulating results in a composition suitable for oral administration.

16. The method of claim 15, wherein said formulating includes formulating said protein with a buffer species.

17. The method of claim 15, wherein said formulating includes formulating said protein with a buffer species comprised of histidine.

18. The method of claim 15, wherein said formulating includes formulating a cytokine.

19. The method of claim 18, wherein said formulating includes a type I interferon.

20. The method of claim 19, wherein said formulating includes formulating a type-I interferon selected from interferon-αa, interferon-β, interferon-ω, and interferon-τ.

21. The method of claim 15, wherein said formulating is comprised of formulating interferon-τ.

22. The method of claim 21, wherein said formulating is comprised of formulating interferon-τ with histidine.

23. The method of claim 15, wherein said formulating comprises formulating with a species effective to bind with said protein to stabilize the protein's tertiary structure.

24. The method of claim 15, wherein said formulating includes formulating said protein and said buffer at a pH value of between about 6-8.

25. A method for selecting a dosage form composition for a protein that achieves protein stabilization for biological activity upon in vivo administration, comprising
selecting a protein for formulation;
preparing solutions of the selected protein or polypeptide in different buffers at different pH values; and
measuring the effect of the buffer on the protein's tertiary structure,
whereby said measuring identifies buffers that result retention of the protein's tertiary structure.

26. The method of claim 25, wherein said selecting comprises selecting a type I interferon.

27. The method of claim 25, wherein said measuring is by spectroscopy.


SEQUENCE LISTING (18 sequences)

SEQ ID NO: 1 - protein, 172 aa, Ovis aries
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 2 - protein, 172 aa, Artificial
  recombinant mature ovine IFNtau, based on SEQ ID NO1.
CYLSERLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 3 - protein, 37 aa, Artificial
  fragment 1-37 of SEQ ID NO1
CYLSRKLMLDARENLKLLDRMNRLSPHSCLQDRKDFG

SEQ ID NO: 4 - protein, 28 aa, Artificial
  fragment 1-28 of SEQ ID NO1
CYLSRKLMLDARENLKLLDRMNRLSPHS

SEQ ID NO: 5 - protein, 26 aa, Artificial
  fragment 2-27 of SEQ ID NO1
YLSRKLMLDARENLKLLDRMNRLSPH

SEQ ID NO: 6 - protein, 37 aa, Artificial
  fragment 1-37 of SEQ ID NO2
CYLSERLMLDARENLKLLDRMNRLSPHSCLQDRKDFG

SEQ ID NO: 7 - protein, 28 aa, Artificial
  fragment 1-28 of SEQ ID NO2
CYLSERLMLDARENLKLLDRMNRLSPHS

SEQ ID NO: 8 - protein, 166 aa, Homo sapiens
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 9 - protein, 166 aa, Artificial
  hybrid interferon-tau/interferon-alpha protein, where residues 1-37 are interferon-tau residues (SEQ ID NO6) and the remaining residues are interferon-alpha
CYLSERLMLDARENLKLLDRMNRLSPHSCLQDRKDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 10 - protein, 166 aa, Artificial
  hybrid interferon-tau/interferon-alpha protein, where residues 1-28 are interferon-tau residues (SEQ ID NO7) and the remaining residues are interferon-alpha
CYLSERLMLDARENLKLLDRMNRLSPHSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 11 - protein, 166 aa, Artificial
  interferon-alpha analog having specific N-terminal residues replaced with interferon-tau residues
CDLPETHSLDNRRTLMLLDRMNRLSPHSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 12 - protein, 166 aa, Artificial
  interferon-alpha analog having specific N-terminal residues replaced with interferon-tau residues
CYLSRKLMLDARENLKLLDRMNRLSPHSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 13 - protein, 166 aa, Artificial
  interferon-alpha analog having specific N-terminal residues replaced with interferon-tau residues
CYLSRTHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 14 - protein, 166 aa, Artificial
  interferon-alpha analog having specific N-terminal residues replaced with interferon-tau residues
CYLSRKLMLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 15 - protein, 166 aa, Artificial
  interferon-alpha analog having specific N-terminal residues replaced with interferon-tau residues
CYLSRKLMLDARENLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 16 - protein, 166 aa, Artificial
  interferon-alpha analog having specific N-terminal residues replaced with interferon-tau residues
CYLSRKLMLDARENLKLLDRMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 17 - protein, 166 aa, Artificial
  interferon-alpha analog having specific N-terminal residues replaced with interferon-tau residues
CDLPEKLMLDARENLKLLDRMNRLSPHSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 18 - protein, 166 aa, Artificial
  interferon-alpha analog having specific N-terminal residues replaced with interferon-tau residues
CDLPETHSLDARENLKLLDRMNRLSPHSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE